(19) United States
(12) Patent Application Publication
(10) Pub. No.: US 2022/0313218 A1
(43) Pub. Date: Oct. 6, 2022

(54) SIGNAL PROCESSING METHOD, READABLE STORAGE MEDIUM, AND ULTRASONIC IMAGING SYSTEM

(71) Applicant: BOE TECHNOLOGY GROUP CO., LTD., Beijing (CN)

(72) Inventors: Zongmin LIU, Beijing (CN); Jijing HUANG, Beijing (CN); Mengjun HOU, Beijing (CN); Qiong WU, Beijing (CN)

(21) Appl. No.: 17/426,713

(22) PCT Filed: Oct. 26, 2020
(86) PCT No.: PCT/CN2020/123647

§ 371 Date: Jul. 29, 2021

(51) Int. Cl. A61B 8/08; A61B 8/14

(52) U.S. Cl. CPC A61B 8/5207; A61B 8/5215; A61B 8/14

(57) ABSTRACT

A signal processing method for performing processing of logarithmic compression with base k on to-be-processed signal (k being greater than 1), comprising: acquiring value of integer part of a result of logarithmic compression corresponding to the to-be-processed signal; calculating fractional part evaluation parameter Q according to the to-be-processed signal and the value of the integer part, Q=d*k^{T-N}, d being value of the to-be-processed signal and greater than 0, N being the value of the integer part, T being a preset constant; evaluating, according to the fractional part evaluation parameter and a preset correspondence table, value M of a fractional part corresponding to the fractional part evaluation parameter, the correspondence table having different fractional part evaluation parameters and values of the fractional part corresponding to the fractional part evaluation parameters; obtaining the result of logarithmic compression according to value N of the integer part and value M of the fractional part.

### TECHNICAL FIELD

The present disclosure relates to the field of data processing, in particular to a signal processing method, a readable storage medium and an ultrasonic imaging system.

### BACKGROUND

In an ultrasonic imaging system, a dynamic range of an ultrasonic echo signal after compensation for absorption attenuation can reach 50 dB or more, while a dynamic range that a display can show is about 20 dB. In order to display the echo signal, it is necessary to perform compression on the echo signal. Linear compression belongs to proportional compression, which may cause a small loss of signal, and logarithmic compression has an effect of stretching small signals and suppressing large signals, and can map weak echo signals with a small dynamic range to a larger output dynamic range.

However, currently used logarithmic compression processing methods are complicated, require a lot of computing resources, and take a relatively long time to perform a computing processing.

### SUMMARY

The present disclosure is intended to solve at least one of the technical problems existing in the prior art, and proposes a signal processing method, a readable storage medium, and an ultrasonic imaging system.

In a first aspect, embodiments of the present disclosure provide a signal processing method for performing a processing of logarithmic compression with a base k on a signal to be processed, where k is greater than 1, and the signal processing method includes:

acquiring a value of an integer part of a result of the logarithmic compression corresponding to the signal to be processed;

calculating a fractional part evaluation parameter Q according to the signal to be processed and the value of the integer part, where Q=d*k^{T-N}, d is a value of the signal to be processed and is greater than 0, N is the value of the integer part, and T is a preset constant;

evaluating, according to the fractional part evaluation parameter and a preset correspondence table, a value M of a fractional part corresponding to the fractional part evaluation parameter, the correspondence table being configured to have different fractional part evaluation parameters and values of the fractional part corresponding to the fractional part evaluation parameters; and

obtaining the result of the logarithmic compression according to the value N of the integer part and the value M of the fractional part.

In some embodiments, k is an integer greater than 1, and the acquiring a value of an integer part of a result of the logarithmic compression corresponding to the signal to be processed includes:

acquiring a t-bit base-k-numeration number corresponding to the value d of the signal to be processed;

determining a highest-order bit having a non-zero value among t bits of the t-bit base-k-numeration number corresponding to the value d of the signal to be processed;

and obtaining the value N of the integer part according to the determined highest-order bit having a non-zero value among the t bits of the t-bit base-k-numeration number corresponding to the value d of the signal to be processed, where N=s−1, and s indicates an ordinal number of the highest-order bit having a non-zero value among the t bits of the t-bit base-k-numeration number corresponding to the value d of the signal to be processed.

In some embodiments, the determining a highest-order bit having a non-zero value among t bits of the t-bit base-k-numeration number corresponding to the value d of the signal to be processed includes:

initializing ‘a’ to t;

determining whether or not an a-th bit of the t-bit base-k-numeration number corresponding to the value d of the signal to be processed has a value of 0;

in response to determining that the a-th bit of the t-bit base-k-numeration number corresponding to the value d of the signal to be processed has a value of 0, updating ‘a’ to a value of ‘a’ minus one, and performing again, with the updated ‘a’, the determining whether or not an a-th bit of the t-bit base-k-numeration number corresponding to the value d of the signal to be processed has a value of 0; and

in response to determining that the a-th bit of the t-bit base-k-numeration number corresponding to the value d of the signal to be processed has a non-zero value, determining the ordinal number s of the highest-order bit having a non-zero value among the t bits of the t-bit base-k-numeration number corresponding to the value d of the signal to be processed to be equal to ‘a’.

In some embodiments, in the correspondence table, a fractional part evaluation parameter having a value of k^{T+(m+1)*10}⁻ⁱ corresponds to the fractional part having a value of m, where m is an integer within a range of [0, 10ⁱ−1], and i is a preset positive integer.

In some embodiments, the evaluating, according to the fractional part evaluation parameter and a preset correspondence table, a value M of a fractional part corresponding to the fractional part evaluation parameter includes:

initializing b to 0;

determining whether or not the fractional part evaluation parameter Q is smaller than a fractional part evaluation parameter corresponding to the fractional part having a value of b in the correspondence table;

in response to determining that the fractional part evaluation parameter Q is smaller than the fractional part evaluation parameter corresponding to the fractional part having a value of b in the correspondence table, determining the value M of the fractional part corresponding to the fractional part evaluation parameter Q to be equal to b; and

in response to determining that the fractional part evaluation parameter Q is equal to or greater than the fractional part evaluation parameter corresponding to the fractional part having a value of b in the correspondence table, updating b to a value of b plus one, and performing again, with the updated b, the determining whether or not the fractional part evaluation parameter Q is smaller than a fractional part evaluation parameter corresponding to the fractional part having a value of b in the correspondence table.

In some embodiments, the obtaining the result of the logarithmic compression according to the value N of the integer part and the value M of the fractional part includes:

adding a product of the value M of the fractional part and 10⁻ⁱ to the value N of the integer part to obtain a result of the adding as the result of the logarithmic compression.

In some embodiments, i has a value of 1.

In some embodiments, k has a value of 2.

In some embodiments, T has a value equal to a number of bits occupied by the signal to be processed.

In a second aspect, embodiments of the present disclosure further provide a readable storage medium having a program stored therein, where when the program is executed, the signal processing method provided in the first aspect is implemented.

In a third aspect, embodiments of the present disclosure further provide an ultrasonic imaging system, including an ultrasonic receiving module having a program stored therein, where when the program is executed, the signal processing method provided in the first aspect is implemented with an echo signal received by the ultrasonic receiving module as the signal to be processed.

In some embodiments, the ultrasonic receiving module includes a field programmable gate array and a receiving chip;

the receiving chip is configured to receive the echo signal transmitted by a ultrasonic probe, amplify the received echo signal, and transmit the amplified echo signal to the field programmable gate array; and the field programmable gate array includes a memory and a processor, the memory stores the program therein, and the processor is configured to execute the program to implement the signal processing method provided in the first aspect with the echo signal as the signal to be processed.

In some embodiments, the ultrasonic imaging system further includes a power module, an ultrasonic transmission module, and an ultrasonic probe;

the power module is configured to supply power to the ultrasonic imaging system;

the ultrasonic transmission module is configured to control the ultrasonic probe to transmit an ultrasonic wave; and

the ultrasonic probe is configured to transmit the ultrasonic wave and generate the echo signal according to a received ultrasonic wave, and transmit the echo signal to the ultrasonic receiving module.

In some embodiments, the ultrasonic imaging system further includes a display module; and

the display module is configured to display data according to the echo signal subjected to a processing of logarithmic compression.

### BRIEF DESCRIPTION OF THE DRAWINGS

FIG. 1 is a flowchart of a signal processing method provided by an embodiment of the present disclosure;

FIG. 2 is a flowchart of an optional implementation method for implementing step S1 in an embodiment of the present disclosure;

FIG. 3 is a flowchart of an optional implementation method for implementing step S102 in an embodiment of the present disclosure;

FIG. 4 is a flowchart of an optional implementation method for implementing step S3 in an embodiment of the present disclosure;

FIG. 5 is a schematic block diagram of a structure of an ultrasonic imaging system provided by an embodiment of the present disclosure;

FIG. 6 is a schematic block diagram of a structure of a power module in an embodiment of the present disclosure; and

FIG. 7 is a schematic block diagram of a structure of an ultrasonic receiving module in an embodiment of the present disclosure.

### DETAILED DESCRIPTION

To make those skilled in the art better understand the technical solution of the present disclosure, a signal processing method, a readable storage medium, and an ultrasonic imaging system provided by the present disclosure will be described in detail below with reference to the accompanying drawings.

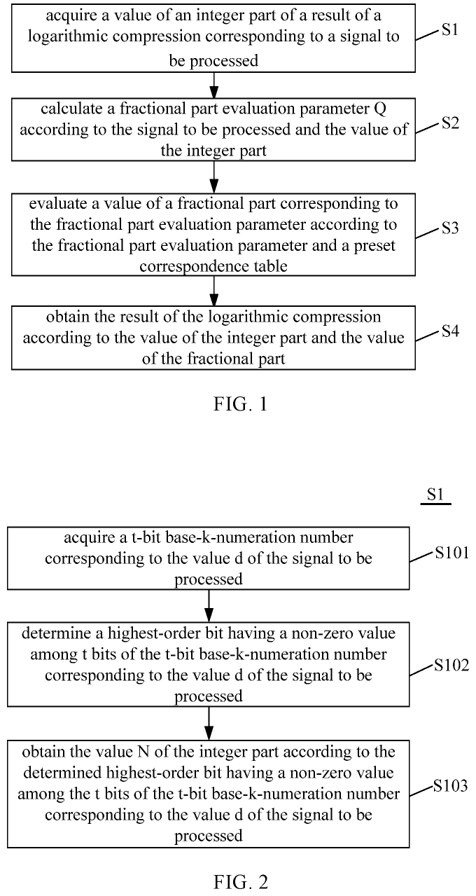

FIG. 1 is a flowchart of a signal processing method provided by an embodiment of the present disclosure. As shown in FIG. 1, the signal processing method is used for performing a processing of logarithmic compression with a base k on a signal to be processed, where k is greater than 1. The signal processing method includes steps S1 to S4.

In step S1, a value of an integer part of a result of the logarithmic compression corresponding to the signal to be processed is acquired.

In an embodiment of the present disclosure, performing a processing of logarithmic compression with a base k on a value d of a signal to be processed refers to performing a logarithmic operation with the base k on the value d of the signal to be processed, that is, solving an approximate value of logₖd and regarding the solved approximate value of logₖd as the result of the logarithmic compression corresponding to the signal to be processed

It should be noted that the signal to be processed in the embodiments of the present disclosure is a digital signal, and the value of the signal to be processed is a value represented by the digital signal.

In step S1, the step of acquiring a value of an integer part of a result of the logarithmic compression corresponding to the signal to be processed is to solve a value of N that can satisfy an inequation k^{N}≤d<k^{N+1}, where N is an integer. The embodiments of the present disclosure do not limit specific technical means to solve the inequation. For example, a specific operational method may be used to obtain the value of N, or a trial and error method may be used to obtain the value of N. Therefore, the technical solution of the present disclosure does not limit specific technical means for obtaining the value N of the integer part in step S1.

In step S2, a fractional part evaluation parameter Q is calculated according to the signal to be processed and the value of the integer part.

The fractional part evaluation parameter Q satisfies the equation Q=d*k^{T-N}, where d is the value of the signal to be processed and is greater than 0, N is the value of the integer part, and T is a preset constant.

Since logₖ d=logₖ (d*k^{-N}*k^{N})=N+logₖ(d*k^{-N}), a value of logₖ (d*k^{-N}) is the same as a part of a value of logₖ d after the decimal point. Therefore, a value of a fractional part of the result of the logarithmic compression may be evaluated by evaluating the value of logₖ (d*k^{-N}). In the case where k has been determined, the value of logₖ (d*k^{-N}) depends on d*k^{-N}, and thus, there is a correspondence between the value of d*k^{-N} and the evaluated value of the fractional part of the result of the logarithmic compression, and there is a correspondence between a value of a product of d*k^{-N} and a set constant k^{T}, namely d*k^{T-N}, and the evaluated value of the fractional part of the result of the logarithmic compression.

In step S3, the value of the fractional part corresponding to the fractional part evaluation parameter is evaluated according to the fractional part evaluation parameter and a preset correspondence table.

The correspondence table is configured to have different fractional part evaluation parameters and values of the fractional part corresponding to the fractional part evaluation parameters.

It can be seen from the above that, there is a correspondence between the value of d*k^{T-N} and the evaluated value of the fractional part of the result of the logarithmic compression, and thus, a correspondence table storing the fractional part evaluation parameters and the values of the fractional part may be generated in advance. Based on the value of d*k^{T-N} and the correspondence data recorded in the correspondence table, the value M of the fractional part corresponding to the fractional part evaluation parameter Q may be evaluated.

In steps S2 and S3, the fractional part evaluation parameter Q is firstly calculated, and then the fractional part evaluation parameter Q is compared with the data recorded in the correspondence table, and thus the value M of the fractional part corresponding to the fractional part evaluation parameter may be quickly evaluated.

Such a processing method of query and comparison based on the correspondence table has a simple operation process and requires less operation resources, and is beneficial to reduce an operation processing time for the process of logarithmic compression and save operation resources.

In some embodiments, a value of k is 2, and a value of T is equal to a number t0 of bits occupied by the signal to be processed. Considering that the operation resources consumed by a division operation may be much greater than the operation resources consumed by a multiplication operation in a digital circuit, the value of T is set to t0. In the case where the number of bits occupied by the signal to be processed is t0, the value of d is an integer within a range of [0, 2^{t0}−1]. In the case where the value of k is 2, the value N of the integer part of log₂d is constantly less than or equal to t0. In the case where the value of T is t0, t0-N≥0 and a result of (t0−N) is an integer, the operation by which the digital circuit calculates the value of d*k^{t0-N} is the multiplication operation, thereby effectively enhancing the operation speed and saving operation resources. It should be noted that T may also take other values in an embodiment of the present disclosure, for example, T=0. The value of T is not limited by the technical solution of the present disclosure.

In step S4, the result of the logarithmic compression is obtained according to the value N of the integer part and the value M of the fractional part.

The result of the logarithmic compression may be divided into two parts: the value N of the integer part and the value M of the fractional part. The number i of digit(s) after the decimal point in the result of the logarithmic compression may be preset. For example, the result of the logarithmic compression is accurate to 1 decimal place, or the result of the logarithmic compression is accurate to 2 decimal places.

The value N of the integer part may reflect digit(s) before the decimal point in the result of the logarithmic compression. The value N of the integer part may be expressed as an integer which may directly express the digit(s) before the decimal point in the result of the logarithmic compression.

The value M of the fractional part may reflect the digit(s) after the decimal point in the result of the logarithmic compression. The value M of the fractional part may be expressed as an integer or a decimal. In the case where the value M of the fractional part is expressed as a decimal, digit(s) after the decimal point in the decimal is the digit(s) after the decimal point in the result of the logarithmic compression. In the case where the value M of the fractional part is expressed as an integer, the digit(s) after the decimal point in the result of the logarithmic compression is digit(s) after the decimal point in a decimal that is obtained by multiplying the integer by 10⁻ⁱ, where i is a positive integer, and indicates the preset number of digit(s) after the decimal point in the result of the logarithmic compression.

In an embodiment of the present disclosure, in the case where the value N of the integer part and the value M of the fractional part are both determined, the result of the logarithmic compression may be obtained by calculation.

In the case where the value N of the integer part and the value M of the fractional part are both expressed in integer form, a product of the value M of the fractional part and 10⁻ⁱ may be added to the value N of the integer part, and a result of the adding is obtained as the result of the logarithmic compression. In the case where the value N of the integer part is expressed in integer form and the value M of the fractional part is expressed in decimal form, the value M of the fractional part may be added to the value N of the integer part, and a result of the adding is obtained as the result of the logarithmic compression.

As an example, in the case where the value N of the integer part is 0, the value M of the fractional part is 3, and the preset number i of digit(s) after the decimal point in the result of the logarithmic compression is equal to 1, the result of the logarithmic compression may be obtained as N+M*10⁻ⁱ=0+3×10⁻¹=0.3. As another example, in the case where the value N of the integer part is 11 and the value M of the fractional part is 0.05, the value N of the integer part and the value M of the fractional part is directly summed, and the result of the logarithmic compression may be obtained as N+M=11.05.

In the digital circuit, it is easier to store and perform operations on an integer type data as compared with a decimal type data. Therefore, in the logarithmic compression processing method provided by the embodiments of the present disclosure, the value of the fractional part is expressed as an integer in some embodiments.

The signal processing method provided by the embodiments of the present disclosure has a simple operation process and requires less operation resources, and is beneficial to reduce an operation processing time for the process of logarithmic compression and save operation resources.

Considering that data in the digital circuit is stored and operated in binary form, in some embodiments, the value of k is 2, such that the digital circuit can implement the signal processing method provided by the embodiments of the present disclosure with fast speed. In the case where the value of k is not 2, the digital circuit may convert the operation process with a base k to an operation process with a base 2 through a base-conversion operation, which also falls within the protection scope of the present disclosure.

FIG. 2 is a flowchart of an optional implementation method for implementing step S1 in an embodiment of the present disclosure. As shown in FIG. 2, in some embodiments, k is an integer greater than 1, and step S1 includes steps S101 to S103.

In step S101, a t-bit base-k-numeration number corresponding to the value d of the signal to be processed is acquired.

In an embodiment of the present disclosure, a range of integers that may be represented by a t-bit number in a numeration system with a base of k is [0, k^{t}−1]. Therefore, in the case where the value of k is determined, the value of t may be set according to a maximum value Dmax of the signal to be processed acquired from previous experience to satisfy k^{t}≥Dmax. At this time, the value d of any signal to be processed is within the range of [0, k^{t}−1].

As an example, in the case where the value of k is 2, if the number of bits occupied by one signal to be processed in the digital circuit is set to t0 in advance and d is a positive integer less than 2′, the value oft may be set to t0.

In step S102, a highest-order bit having a non-zero value among t bits of the t-bit base-k-numeration number corresponding to the value d of the signal to be processed is determined.

In step S103, the value N of the integer part is obtained according to the determined highest-order bit having a non-zero value among the t bits of the t-bit base-k-numeration number corresponding to the value d of the signal to be processed.

The value N of the integer part is equal to s−1, where s indicates the ordinal number of the highest-order bit having a non-zero value among the t bits of the t-bit base-k-numeration number corresponding to the value d of the signal to be processed, which is determined in step S102.

Taking a case where the value of k is 2, the value oft is 5, and the value d of the signal to be processed is 9 as an example, in step S101, a 5-bit binary number corresponding to d=9 may be obtained as “01001”, a value of a first bit of “01001” is 1, a value of a second bit of “01001” is 0, a value of a third bit of “01001” is 0, a value of a fourth bit of “01001” is 1, and a value of a fifth bit of “01001” is 0; in step S102, a highest-order bit having a non-zero value among “01001” is the fourth bit, that is, s=4; in step S103, it may be obtained by calculation that N=s−1=4−1=3. That is, when the processing of logarithmic compression with a base 2 is performed on the value d, that is equal to 9, of the signal to be processed, the value M of the integer part of the result of the logarithmic compression corresponding thereto is 3.

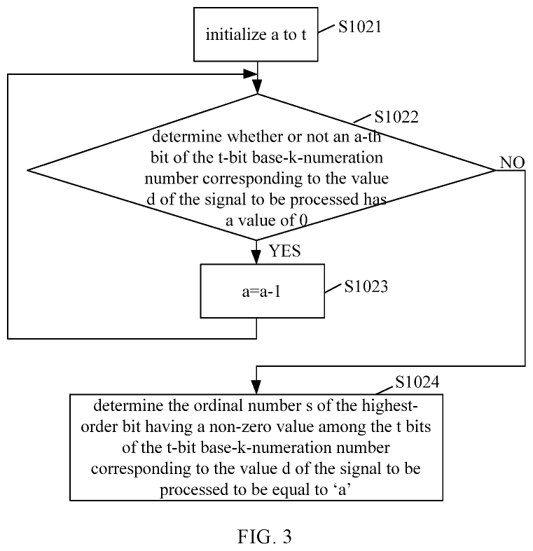

FIG. 3 is a flowchart of an optional implementation method for implementing step S102 in an embodiment of the present disclosure. As shown in FIG. 3, in some embodiments, the ordinal number s of the highest-order bit having a non-zero value among the t bits of the t-bit base-k-numeration number corresponding to the value d of the signal to be processed may be determined by data shifting and comparison. Step S102 includes steps S1021 to S1024.

In step S1021, ‘a’ is initialized to t.

In step S1022, it is determined whether or not an a-th bit of the t-bit base-k-numeration number corresponding to the value d of the signal to be processed has a value of 0.

Upon it is determined that the a-th bit of the t-bit base-k-numeration number corresponding to the value d of the signal to be processed has a value of 0, it proceeds to step S1023; and upon it is determined that the a-th bit of the t-bit base-k-numeration number corresponding to the value d of the signal to be processed has a non-zero value, it proceeds to step S1024.

In step S1023, ‘a’ is updated to a result of ‘a’ minus one.

Upon step S1023 is completed, step S1022 is performed again with the updated ‘a’.

In step S1024, it is determined that the ordinal number s of the highest-order bit having a non-zero value among the t bits of the t-bit base-k-numeration number corresponding to the value d of the signal to be processed is equal to ‘a’.

In the embodiments of the present disclosure, the ordinal number s of the highest-order bit having a non-zero value, among the t bits of the t-bit base-k-numeration number corresponding to the value d of the signal to be processed, is determined by data shifting and comparison, which has a simple operation process and requires less operation resources, and is beneficial to reduce an operation processing time for the process of logarithmic compression and save operation resources.

It should be noted that the above-mentioned data shifting and comparison is a possible implementation in the embodiments of the present disclosure, and the technical solution of the present disclosure is not limited thereto. In an embodiment of the present disclosure, other methods may also be used to determine the ordinal number s of the highest-order bit having a non-zero value among the t bits of the t-bit base-k-numeration number.

In some embodiments, in the correspondence table, the fractional part evaluation parameter having a value of k^{T+(m+1)*10}⁻ⁱ corresponds to the fractional part having a value of m, where m is an integer within a range of [0, 10ⁱ−1], i indicates the preset number of digit(s) after the decimal point in the result of the logarithmic compression, and i is a positive integer.

In some embodiments, i has a value of 1, that is, the result of the logarithmic compression is accurate to one decimal place, and this accuracy may meet requirements of data compression processing in some scenarios.

In an embodiment of the present disclosure, the correspondence table only stores the correspondences between 10ⁱ different fractional part evaluation parameters and 10¹ values of the fractional part, in which the fractional part evaluation parameter having a value of k^{T+(m+1)*10}⁻ⁱ corresponds to the fractional part having a value of m. The amount of data stored in the correspondence table may be greatly reduced by such a manner of storing only the correspondences of some threshold points.

Table 1 is a correspondence table by taking a case where the value of k is 2, the value of T is 0, and the value of i is 1 as an example, as shown below:

TABLE 1

| k^{T+(m+1)*10-i} | 2^{0.1} | 2^{0.2} | 2^{0.3} | 2^{0.4} | 2^{0.5} | 2^{0.6} | 2^{0.7} | 2^{0.8} | 2^{0.9} | 2¹ |
|---|---|---|---|---|---|---|---|---|---|---|
| m | 0 | 1 | 2 | 3 | 4 | 5 | 6 | 7 | 8 | 9 |

Table 2 is a correspondence table by taking a case where the value of k is 2, the value of T is t0, and the value of i is 1 as an example, as shown below:

TABLE 2

| k^{T+(m+1)*10-i} | 2^{0.1+t0} | 2^{0.1+t0} | 2^{0.3+t0} | 2^{0.4+t0} | 2^{0.5+t0} | 2^{0.6+t0} | 2^{0.7+t0} | 2^{0.8+t0} | 2^{0.9+t0} | 2^{1+t0} |
|---|---|---|---|---|---|---|---|---|---|---|
| m | 0 | 1 | 2 | 3 | 4 | 5 | 6 | 7 | 8 | 9 |

It can be seen from Table 1 and Table 2 that, in the case where the value of i is 1, the value of m is in the range of [0, 9] and there are 10 different values of m in total, the correspondence tables shown in Table 1 and Table 2 only need to have 10 pieces of correspondence relationships, and the amount of data stored in the correspondence tables is small.

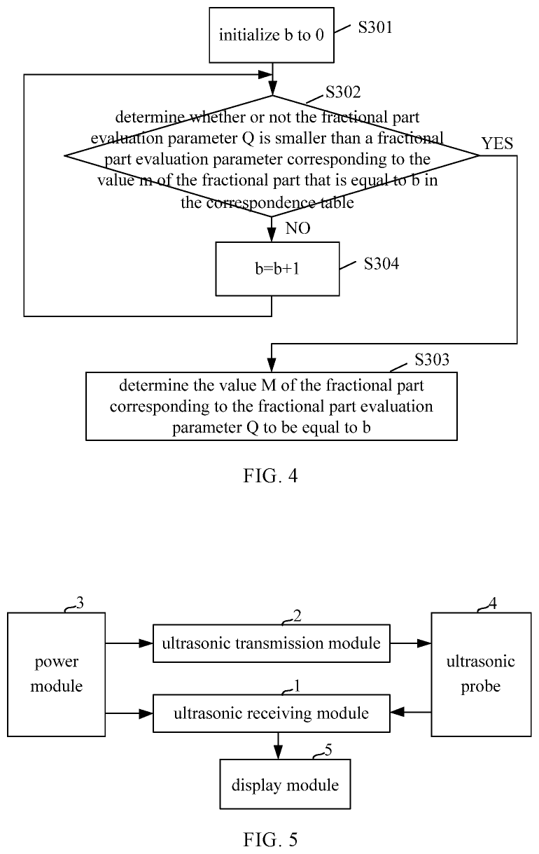

FIG. 4 is a flowchart of an optional implementation method for implementing step S3 in an embodiment of the present disclosure. As shown in FIG. 4, in some embodiments, step S3 includes steps S301 to S304.

In step S301, b is initialized to 0.

In step S302, it is determined whether or not the fractional part evaluation parameter Q is smaller than a fractional part evaluation parameter corresponding to the value m of the fractional part that is equal to b in the correspondence table.

In step S302, the fractional part evaluation parameter k^{T+(m+1)*10}⁻ⁱ corresponding to the value m of the fractional part that is equal to b is firstly queried, and then the fractional part evaluation parameter Q calculated in step S2 is compared with the queried fractional part evaluation parameter k^{T+(m+1)*10}⁻ⁱ.

Upon it is determined that the fractional part evaluation parameter Q is smaller than the fractional part evaluation parameter k^{T+(m+1)*10}⁻ⁱ corresponding to the value m of the fractional part that is equal to b in the correspondence table, it proceeds to step S303. Upon it is determined that the fractional part evaluation parameter Q is equal to or greater than the fractional part evaluation parameter corresponding to the fractional part having a value of b in the correspondence table, it proceeds to step S304.

In step S303, the value M of the fractional part corresponding to the fractional part evaluation parameter Q is determined to be equal to b.

In step S304, b is updated to a result of b plus one.

Upon S304 is completed, step S302 is performed again with the updated b.

In the embodiments of the present disclosure, the value M of the fractional part of the result of the logarithmic compression is determined by querying the correspondence table and comparing with the queried data, which has a simple operation process and requires less operation resources, and is beneficial to reduce an operation processing time for the process of logarithmic compression and save operation resources.

The signal processing method provided by the embodiments of the present disclosure has a simple operation process and requires less operation resources, and is beneficial to reduce an operation processing time for the process of logarithmic compression and save operation resources. The signal processing method can be used to perform the processing of logarithmic compression on data in different application scenarios. For example, when the signal processing method is applied to the ultrasonic imaging system, the signal processing method can be used to perform the processing of logarithmic compression on the echo signal to reduce the dynamic range of the echo signal, such that a display system can display the echo signal.

FIG. 5 is a schematic block diagram of a structure of an ultrasonic imaging system provided by an embodiment of the present disclosure. As shown in FIG. 5, the ultrasonic imaging system includes an ultrasonic receiving module 1 having a program stored therein, and when the program is executed, the signal processing method provided by any of the above embodiments is implemented with an echo signal received by the ultrasonic receiving module as the signal to be processed. The signal processing method may be described with reference to the content in the previous embodiments, which will not be repeated here.

In some embodiments, the ultrasonic imaging system further includes a power module 3, an ultrasonic transmission module 2, and an ultrasonic probe 4. The power module 3 is configured to supply power to various functional modules in the ultrasonic imaging system, for example, to the ultrasonic transmission module 2 and the ultrasonic receiving module 1. The ultrasonic transmission module 2 is configured to control the ultrasonic probe 4 to transmit an ultrasonic wave. The ultrasonic probe 4 is configured to transmit the ultrasonic wave and generate the echo signal according to a received ultrasonic wave, and transmit the echo signal to the ultrasonic receiving module 1.

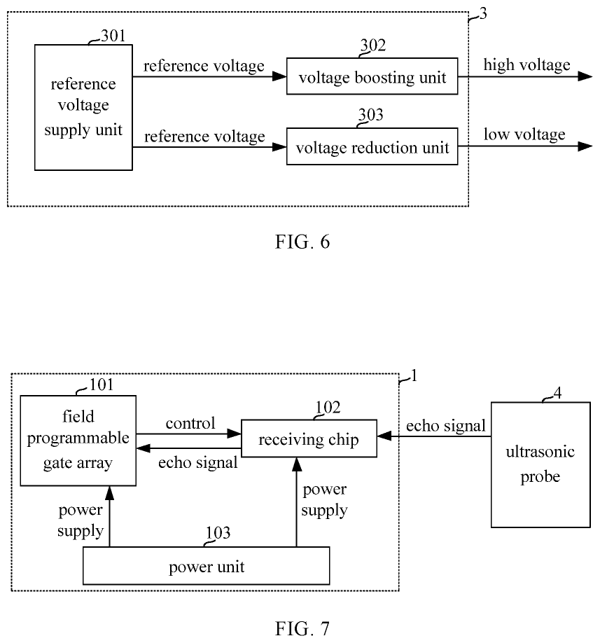

FIG. 6 is a schematic block diagram of a structure of a power module in an embodiment of the present disclosure. As shown in FIG. 6, the power module 3 includes a reference voltage supply unit 301, a voltage boosting unit 302, and a voltage reduction unit 302. The reference voltage supply unit 301 is configured to provide a reference voltage (for example, ±15V) to the voltage boosting unit and the voltage reduction unit. The voltage boosting unit 302 is configured to boost the reference voltage and output a high voltage (for example, ±100V). The voltage reduction unit 303 is configured to reduce the reference voltage and output a low voltage (for example, ±10V, ±5V, ±3.3V).

In some embodiments, the ultrasonic imaging system further includes a display module 5. The display module 5 is configured to display data according to the echo signal subjected to the processing of logarithmic compression.

FIG. 7 is a schematic block diagram of a structure of an ultrasonic receiving module in an embodiment of the present disclosure. As shown in FIG. 7, in some embodiments, the ultrasonic receiving module 1 includes a field programmable gate array (FPGA) 101 and a receiving chip 102. The receiving chip 2 is configured to receive the echo signal transmitted by the ultrasonic probe, amplify the received echo signal, and transmit the amplified echo signal to the field programmable gate array 101. The field programmable gate array 101 includes a memory and a processor, the memory stores the program therein, and the processor is configured to execute the program to implement the signal processing method provided in any of the above embodiments with the echo signal as the signal to be processed.

In addition, the field programmable gate array 101 may be further configured to control the receiving chip 102 to operate, and perform an processing such as beamforming, dynamic filtering, and envelope detection on the echo signal transmitted by the receiving chip 102 before performing the processing of logarithmic compression on the echo signal.

Considering that the amount of operations of the ultrasonic receiving module 1 is relatively large and a power supply requirement thereof is high, a dedicated power unit 103 is also provided in the ultrasonic receiving module 1, and the power unit is configured to supply power to the field programmable gate array 101 and the receiving chip 102.

An embodiment of the present disclosure further provides a readable storage medium having a program stored therein, and when the program is executed, the signal processing method provided in any of the above embodiments is implemented.

Those of ordinary skill in the art can understand that all or some of the steps in the method and the functional modules/units in the device disclosed above may be implemented as software, firmware, hardware, and appropriate combinations thereof. In the hardware implementation, the division for the functional modules/units mentioned in the above description does not necessarily correspond to the division for physical components. For example, one physical component may have multiple functions, or one function or step may be performed by several physical components in cooperation. Some physical components or all physical components may be implemented as software executed by a processor such as a central processing unit, a digital signal processor, or a microprocessor, or as hardware, or as an integrated circuit such as an application specific integrated circuit. Such software may be distributed on a computer-readable medium, and the computer-readable medium may include a computer storage medium (or a non-transitory medium) and a communication medium (or a transitory medium). As is well known to those of ordinary skill in the art, the term “computer storage medium” includes volatile medium and non-volatile medium, removable medium and non-removable medium implemented in any method or technic for storing information (such as computer-readable instruction, data structure, program module, or other data). The computer storage medium includes, but is not limited to, RAM, ROM, EEPROM, flash memory or other memory technologies, CD-ROM, digital versatile disk (DVD) or other optical disk storage, magnetic cassette, magnetic tape, magnetic disk storage or other magnetic storage device, or any other medium that may be used to store desired information and may be accessed by a computer. In addition, as is well known to those of ordinary skill in the art, the communication medium usually contains computer-readable instruction, data structure, program module, or other data in a modulated data signal such as carrier wave or other transmission mechanism, and may include any information delivery medium.

Example embodiments have been disclosed herein, and although specific terms have been adopted, the specific terms are only used to be interpreted as and should only be interpreted as general descriptive meanings, and are not used for the purpose of limitation. In some instances, it is conceivable to those skilled in the art that, unless expressly indicated otherwise, features, characteristics, and/or elements described in combination with a specific embodiment may be used alone, or may be used in combination with features, characteristics, and/or elements described in combination with other embodiments. Therefore, those skilled in the art will understand that various changes in form and detail can be made without departing from the scope of the present disclosure as set forth by the appended claims.

1. A signal processing method for performing a processing of logarithmic compression with a base k on a signal to be processed, k being greater than 1, the signal processing method comprising:
acquiring a value of an integer part of a result of the logarithmic compression corresponding to the signal to be processed;
calculating a fractional part evaluation parameter Q according to the signal to be processed and the value of the integer part, where Q=d*k^{T-N}, d is a value of the signal to be processed and is greater than 0, N is the value of the integer part, and T is a preset constant;
evaluating, according to the fractional part evaluation parameter and a preset correspondence table, a value M of a fractional part corresponding to the fractional part evaluation parameter, the correspondence table being configured to have different fractional part evaluation parameters and values of the fractional part corresponding to the fractional part evaluation parameters; and
obtaining the result of the logarithmic compression according to the value N of the integer part and the value M of the fractional part.

2. The signal processing method according to claim 1, wherein k is an integer greater than 1, and the acquiring a value of an integer part of a result of the logarithmic compression corresponding to the signal to be processed comprises:
acquiring a t-bit base-k-numeration number corresponding to the value d of the signal to be processed;
determining a highest-order bit having a non-zero value among t bits of the t-bit base-k-numeration number corresponding to the value d of the signal to be processed; and
obtaining the value N of the integer part according to the determined highest-order bit having a non-zero value among the t bits of the t-bit base-k-numeration number corresponding to the value d of the signal to be processed, where N=s−1, and s indicates an ordinal number of the highest-order bit having a non-zero value among the t bits of the t-bit base-k-numeration number corresponding to the value d of the signal to be processed.

3. The signal processing method according to claim 2, wherein the determining a highest-order bit having a non-zero value among t bits of the t-bit base-k-numeration number corresponding to the value d of the signal to be processed comprises:
initializing ‘a’ to t;
determining whether or not an a-th bit of the t-bit base-k-numeration number corresponding to the value d of the signal to be processed has a value of 0;
in response to determining that the a-th bit of the t-bit base-k-numeration number corresponding to the value d of the signal to be processed has a value of 0, updating ‘a’ to a value of ‘a’ minus one, and performing again, with the updated ‘a’, the determining whether or not an a-th bit of the t-bit base-k-numeration number corresponding to the value d of the signal to be processed has a value of 0; and
in response to determining that the a-th bit of the t-bit base-k-numeration number corresponding to the value d of the signal to be processed has a non-zero value, determining the ordinal number s of the highest-order bit having a non-zero value among the t bits of the t-bit base-k-numeration number corresponding to the value d of the signal to be processed to be equal to ‘a’.

4. The signal processing method according to claim 1, wherein in the correspondence table, a fractional part evaluation parameter having a value of k^{T+(m+1)*10}⁻ⁱ corresponds to the fractional part having a value of m, where m is an integer within a range of [0, 10ⁱ−1], and i is a preset positive integer.

5. The signal processing method according to claim 4, wherein the evaluating, according to the fractional part evaluation parameter and a preset correspondence table, a value M of a fractional part corresponding to the fractional part evaluation parameter comprises:
initializing b to 0;
determining whether or not the fractional part evaluation parameter Q is smaller than a fractional part evaluation parameter corresponding to the fractional part having a value of b in the correspondence table;
in response to determining that the fractional part evaluation parameter Q is smaller than the fractional part evaluation parameter corresponding to the fractional part having a value of b in the correspondence table, determining the value M of the fractional part corresponding to the fractional part evaluation parameter Q to be equal to b; and
in response to determining that the fractional part evaluation parameter Q is equal to or greater than the fractional part evaluation parameter corresponding to the fractional part having a value of b in the correspondence table, updating b to a value of b plus one, and performing again, with the updated b, the determining whether or not the fractional part evaluation parameter Q is smaller than a fractional part evaluation parameter corresponding to the fractional part having a value of b in the correspondence table.

6. The signal processing method according to claim 4, wherein the obtaining the result of the logarithmic compression according to the value N of the integer part and the value M of the fractional part comprises:
adding a product of the value M of the fractional part and 10⁻ⁱ to the value N of the integer part to obtain a result of the adding as the result of the logarithmic compression.

7. The signal processing method according to claim 4, wherein i has a value of 1.

8. The signal processing method according to claim 1, wherein k has a value of 2.

9. The signal processing method according to claim 8, wherein T has a value equal to a number of bits occupied by the signal to be processed.

10. A readable storage medium having a program stored therein, wherein when the program is executed, the signal processing method according to claim 1 is implemented.

11. An ultrasonic imaging system, comprising: an ultrasonic receiving module having a program stored therein, wherein when the program is executed, the signal processing method according to claim 1 is implemented with an echo signal received by the ultrasonic receiving module as the signal to be processed.

12. An ultrasonic imaging system, comprising: an ultrasonic reviving module, wherein the ultrasonic receiving module comprises a field programmable gate array and a receiving chip;
the receiving chip is configured to receive an echo signal transmitted by a ultrasonic probe, amplify the received echo signal, and transmit the amplified echo signal to the field programmable gate array; and
the field programmable gate array comprises a memory and a processor, the memory stores a program therein, and the processor is configured to execute the program to implement the signal processing method according to claim 1 with the echo signal as the signal to be processed.

13. The ultrasonic imaging system according to claim 11, further comprising: a power module, an ultrasonic transmission module, and an ultrasonic probe;
the power module is configured to supply power to the ultrasonic imaging system;
the ultrasonic transmission module is configured to control the ultrasonic probe to transmit an ultrasonic wave; and
the ultrasonic probe is configured to transmit the ultrasonic wave and generate the echo signal according to a received ultrasonic wave, and transmit the echo signal to the ultrasonic receiving module.

14. The ultrasonic imaging system according to claim 11, further comprising a display module,
wherein the display module is configured to display data according to the echo signal subjected to a processing of logarithmic compression.
